(19) United States
(12) Patent Application Publication
(10) Pub. No.: US 2020/0306555 A1
(43) Pub. Date: Oct. 1, 2020

(54) Compact Portable Body Tanning and Light Therapy Apparatus

(71) Applicant: John Oscar Alexander Ebbesson, Visby (SE)

(72) Inventor: John Oscar Alexander Ebbesson, Visby (SE)

(21) Appl. No.: 16/372,085

(22) Filed: Apr. 1, 2019

(51) Int. Cl. A61N 5/06

(52) U.S. Cl. CPC A61N 5/0614; A61N 5/0618; A61N 2005/0637; A61N 2005/0634; A61N 2005/0633

(57) ABSTRACT

The present invention provides an apparatus and method for self-tanning or light therapy treatment at home using a device capable of emitting ultraviolet radiation to achieve a tan on the user's body. The invention can also be configured for use in light therapy applications. A complete tan is achieved by lying on a flat surface underneath the apparatus emitting ultraviolet radiation and controlling forward and backward movement of the apparatus along a flat surface by either manually sliding the apparatus along the body or by means of a motorized drive system to focus the ultraviolet radiation on different parts of the body.

### FIELD OF INVENTION

The present invention relates to tanning the skin of a human body, specifically an apparatus and method for self-tanning at home using a device capable of emitting ultraviolet radiation to achieve a tan on the user's body.

### BACKGROUND OF THE INVENTION

In-home tanning solutions have not been successful in reaching broad consumer acceptance due primarily to the large equipment and expense necessary to achieve a natural looking tan one would normally obtain from a tanning bed using ultraviolet (“UV”) light-emitting lamps or bulbs. Users of these devices normally must travel to tanning salons or spas to use such equipment. There is an absence of affordable and compact solutions for achieving a tan using UV lamps conveniently at home.

A need exists for a body tanning device that is easy to use at home, have a form much smaller than a traditional tanning bed that can accommodate a relatively small space when in use, and have features allowing it to be easily stored away when not in use.

Several attempts have been made to solve the problems outlined above in the field of tanning devices, including those found in the following prior art: U.S. Pat. No. 7,001,414 to Unvert et. al., U.S. Pat. No. 4,740,707 to Thaw, U.S. Pat. No. 7,641,678 to Mewissen-Scholberg et. al., U.S. Pat. Pub. No. 2014/0031906 to Brezinski, U.S. Pat. No. 5,478,668 to Cooper et. al., and U.S. Pat. No. 4,989,600 to Collier U.S. Pat. Pub. No. 2008/0306575 to Schiavon, to Dutch Patent App. No. NL 9,000,302 to Ultrasun Professional BV, and to UK Patent No. GB2,201,502 to MacFarlane. None of these prior art inventions and patents, either individually or in combination, describe the present invention as claimed.

### SUMMARY OF THE INVENTION

The objects of the present invention are to provide a safe, convenient, and effective apparatus for achieving an even tan on a user's body.

An additional object of the present invention is to provide an apparatus for convenient in-home head-to-toe tanning that does not take up as much space as a traditional tanning bed.

An additional object of the present invention is to provide an apparatus that is easily collapsible for convenient storage and portability when not in use.

An additional object of the present invention is to provide a safe and easy method for achieving a tan on a human body at home by the manual or automated function of the apparatus and in a shorter amount of time compared to preexisting body tanning methods.

An additional object of the present invention is to provide an apparatus for allowing a plurality of different types of bulbs to be used having therapeutic purposes.

The present invention fulfills the above and other objects by providing an apparatus comprising a plurality of panels, each having an exterior and a recessed interior, the panels forming an arc shape defining a space where a person (i.e. “user”) can lie under and having means of connecting to an electrical source such as a wall outlet. The panels can be curved to form a smooth, rounded arc, or can be straight to form a more squared arc above the user. The panels are connected by hinged joints allowing the apparatus to be folded inward and collapsed for storage purposes when not in use. At least one panel has a handle attached to its exterior side for easy carrying. The recessed interior portions of the panels contain housing means for a plurality of bulbs capable of emitting ultraviolet radiation. These bulbs can be of any type capable of emitting ultraviolet radiation including incandescent, fluorescent, LED, or other lighting technology known in the art. The ends of the outer panels that touch the floor (or other horizontal flat surface) have a smoothed finish that may be made from chromium plating, stainless steel, or a layer of fabric, or other means known in the art allowing the apparatus to easily slide along a flat surface. Alternatively, wheels may be affixed to the ends of the apparatus to roll the device along a flat surface. In the preferred embodiment of the invention, the wheels are made to be retractable into a recess in the ends of the housing by means known in the art. In other embodiments, the wheels may be fixed or as a detachable optional feature. The preferred embodiment having wheels may be connected to a controllable motorized drive system allowing the apparatus to be programmed to roll forward and backward along the floor. The apparatus may be manufactured from any lightweight metal or plastic polymer. This would ideally allow for manufacture and modest cost.

Another contemplated use of the invention is for use as a light therapy device, using different types of bulbs besides UV. In another preferred embodiment, the panels would have alternative UV and other bulbs, and the unit would have a toggle switch to change back and forth between UV bulbs for tanning and bulbs designed for therapeutic treatment known in the field of light therapy.

The above and other objects, features and advantages of the present invention should become more readily apparent to those skilled in the art upon a reading of the following detailed description in conjunction with the drawings, wherein there is shown and described illustrative embodiments of the invention.

### BRIEF DESCRIPTION OF THE DRAWINGS

In the following detailed description, reference will be made to the attached drawings in which:

FIG. 1 is an isometric view of the preferred embodiment of an apparatus for a portable tanning machine as claimed in the present invention, having curved panels forming a smooth arc shape and having wheels for rolling across a flat surface;

FIG. 2 is a front view showing the preferred embodiment of the apparatus of FIG. 1;

FIG. 3 is a side view showing the preferred embodiment of the apparatus of FIG. 1 showing the general proportion of the invention to a user of average adult height;

FIG. 4 is a top view showing the preferred embodiment of the apparatus of FIG. 1;

FIG. 5 is an isometric view of an embodiment of an apparatus for a portable tanning machine as claimed in the present invention having straight panels, and showing an example of the preferred method of use;

### DESCRIPTION OF THE PREFERRED EMBODIMENTS

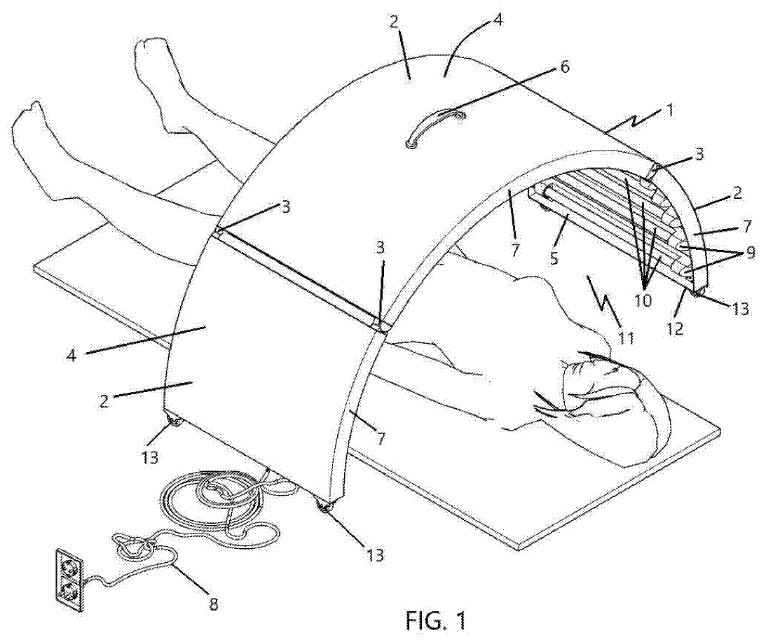

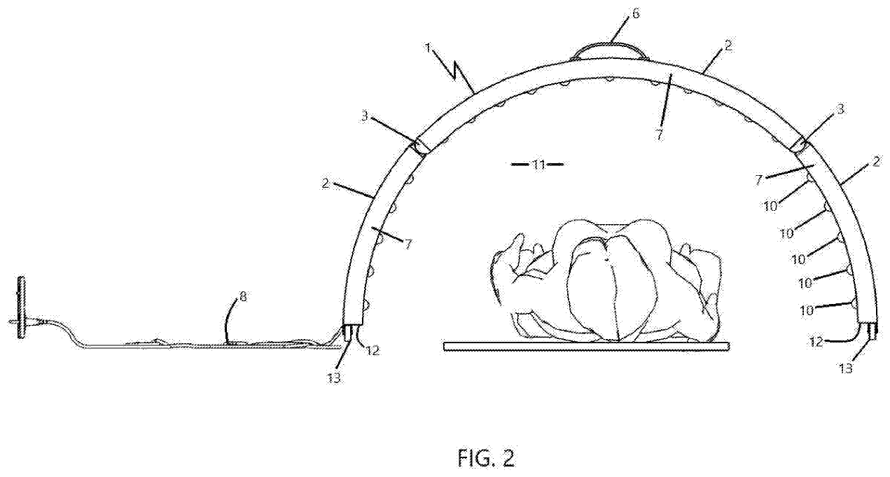

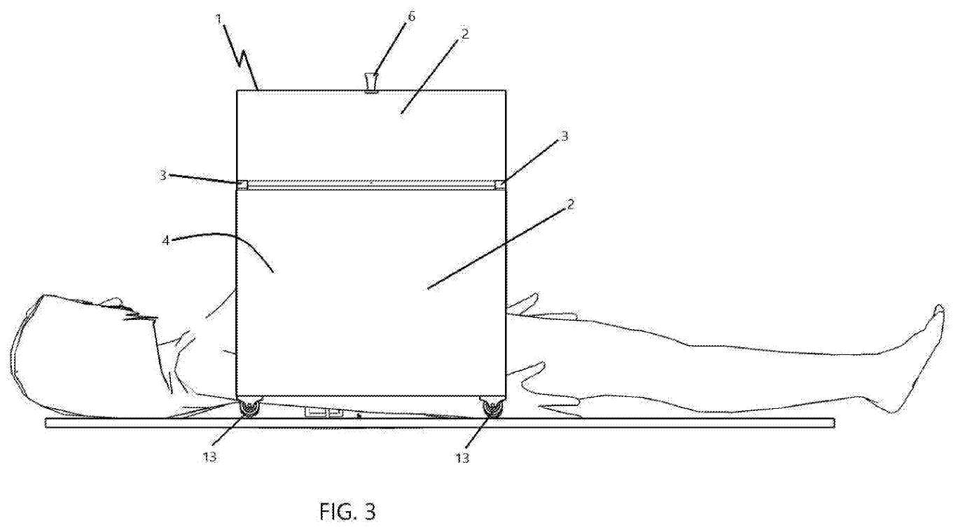

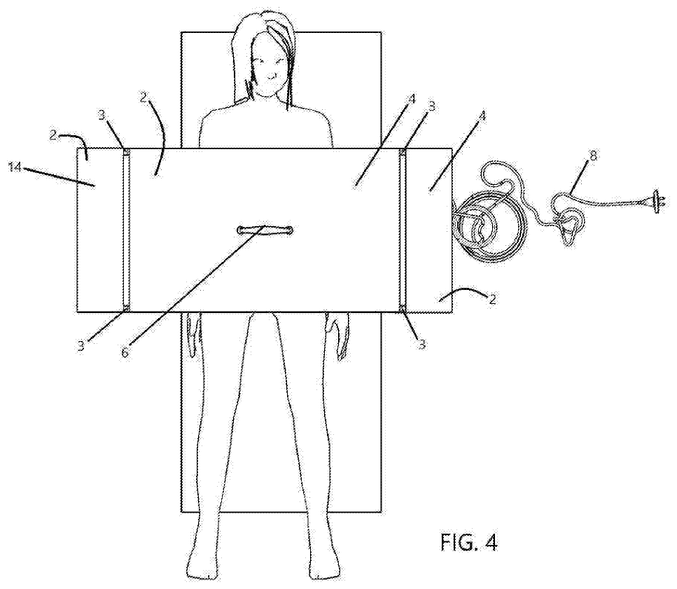

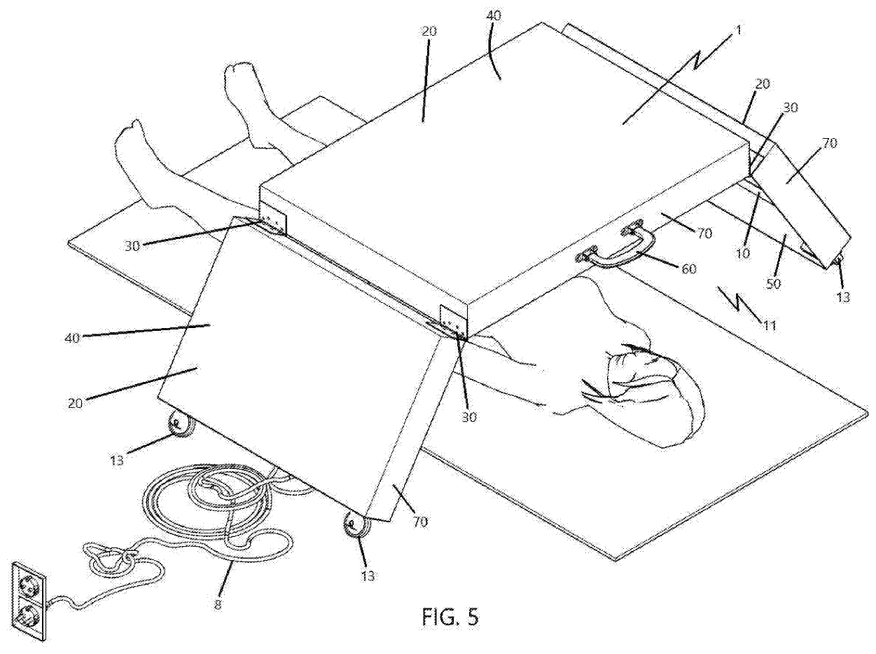

With reference to the drawings, the present invention provides an apparatus and method for self-tanning at home using a device capable of emitting ultraviolet radiation to achieve a tan on the user's body. In FIG. 1, an isometric view of the preferred embodiment is illustrated. The invention has a plurality of curved panels (as shown FIGS. 1, 2, 3, and 4) that are connected to each other with hinged joints forming an arc and defining a space (11) where a user can lay as shown in FIGS. 1, 2, 3, and 5). Alternatively, the plurality of panels (2) may each be straight rather than curved, forming a more squared arc, as shown in FIG. 5. The optimal width of each panel is approximately 0.5 meters and total dimensions of the preferred embodiment of the invention as seen in the figures is between 2.5 and 4 feet long and 1.5 to 2.5 feet wide, in order to achieve the best compromise between shorter tanning time, compactness and portability.

When not in use, the apparatus can be easily collapsed by folding in the panels at the hinged joints (3). This makes for easier and convenient storage at home. At least one handle (6, 60) is affixed to the panel exterior for aid in carrying. The handle can be located either on the top face of the panel exterior (4, 40) as shown in FIG. 1 or on a side face (7, 70) as shown in FIG. 5, or other convenient location on the exterior of the panel.

In the preferred embodiment and best mode of the invention, the panels contain a recessed housing for a multitude of bulbs (10). These bulbs are capable of emitting ultraviolet light to achieve a tan on a user's body. The preferred minimum number of bulbs to achieve the best tan is sixteen, but the apparatus can have more or less than this number of bulbs. The housing (5) of the bulbs (10) can be designed so that the bulbs run either lengthwise or widthwise along the panels (2, 20). In the preferred embodiment, the bulbs (10) are configured to run widthwise, as shown in FIG. 1. The design of the panels (2, 20) as shown in the figures allow for immediate access to the housing (9) to change out bulbs or service the housing as necessary.

The outer ends (12) of the apparatus have wheels (13) that allow the invention to be rolled forward or backward along a flat surface, as shown in FIGS. 1, 2, 3, 4, and 5). The wheels (13) may either be affixed to the ends or can be designed to be removable as desired, and can be constructed and affixed to the invention by any means known in the art. It is further contemplated that the wheels (13) may be connected to a motorized drive system (not shown in the figures) controllable by the user and can be programmed to roll the apparatus (1) forward or backward along the user's body at set time intervals to achieve a tan by means known in the art. Motorized drive systems known in the art are relatively compact and can be housed in the outer panels (6) or any location on the invention (1) deemed desirable for manufacturing.

Alternatively, the ends (12) of the outer panels that contact the floor may have a smooth finish (not shown in the drawings) for ease of gliding along said flat surface. This smooth finish can be achieved either by a chromium plated layer, a stainless-steel layer, a layer of smooth fabric, or other means known in the art for achieving smooth gliding along the floor or a flat surface.

A perfect tan can be achieved by the user of the apparatus by laying in the space underneath the panels and manually sliding the apparatus up or down along the floor along the user's body, about ten minutes at each interval. Whether the panels are curved or straight, a user would only have to make two passes (up and back down) at most with the invention to achieve a perfect tan.

In another embodiment of the invention, the bulbs (10) used in the invention (1) can be designed for providing light therapy rather than for tanning. Several different types of lights are known in the art for providing light therapy, such as dawn simulators, natural spectrum light bulbs, and bluewave light. The apparatus can be designed to accept two series of bulbs, for both body tanning and light therapy applications, with a toggle switch or other means of allowing the user to select either setting.

The figures shown herein display an insulated electric cord (8) as one example of supplying power to the apparatus. This is not meant to be a limiting feature, and other means of supplying power to the invention known in the art are contemplated, such as rechargeable or replaceable battery power.

It is to be understood that while the preferred embodiments of the invention are illustrated, it is not to be limited to the specific forms or arrangements as described and shown. It will be apparent to those skilled in the art that various changes may be made without departing from the scope of the invention and the invention is not to be considered limited to what is shown and described in the specification and drawings.

1. A compact body tanning apparatus comprising:
a plurality of panels having an exterior and a recessed interior, the panels being either curved or straight and forming an arc defining a space where a person can lie under on a flat surface, and having two outer ends that make contact with said flat surface;
each said panel being connected by hinged joints allowing the apparatus to be folded inward for portability and storage;
the outer ends having a smooth finish enabling said apparatus to glide along said flat surface;
at least one handle located on said exterior of said panels;
means for providing electrical power to said apparatus; and
at least one plurality of bulbs mounted to the recessed interior space of each panel, said bulbs either being capable of emitting ultraviolet radiation for body tanning, or capable for use in light therapy applications.

2. The apparatus of claim 1 wherein the apparatus has means of selecting between multiple series of bulbs configured for either tanning or light therapy applications.

3. A compact body tanning apparatus comprising:
a plurality of panels having an exterior and a recessed interior, the panels being either curved or straight and forming an arc defining a space where a person can lie under on a flat surface, and having two outer ends that make contact with said flat surface;
each said panel being connected by hinged joints allowing the apparatus to be folded inward for portability and storage;
a plurality of wheels attached to the two outer ends of the apparatus allowing the apparatus to roll forward or backward along said flat surface;
at least one handle located on said exterior of said panels;
means for providing electrical power to said apparatus; and
at least one series of bulbs mounted to the recessed interior space of each panel, each said series of bulbs either being capable of emitting ultraviolet radiation for body tanning, or capable for use in light therapy applications.

4. The compact body tanning apparatus of claim 3 whereby the plurality of wheels are either fixed, detachable, or retractable into a recess on the outer ends.

5. The compact body tanning apparatus of claim 3, whereby the plurality of wheels are connected to a motorized drive control system capable of being programmed to move forward or backward along a flat surface.

6. The compact body tanning apparatus of claim 1 or 3 wherein the apparatus has means of selecting between multiple series of bulbs configured for either tanning or light therapy applications.

7. A method of tanning a human body comprising:
providing a compact body tanning apparatus according to claim 1;
lying on a flat surface underneath the tanning apparatus;
emitting ultraviolet radiation from the plurality of bulbs, wherein said ultraviolet radiation effects tanning of the skin; and
manually sliding the apparatus forward or backward along a flat surface to focus the ultraviolet radiation on different parts of the body to achieve a complete tan or as desired.

8. A method of tanning a human body comprising:
providing a compact body tanning apparatus according to claim 3 or 5;
lying on a flat surface underneath the apparatus;
emitting ultraviolet radiation from the plurality of bulbs, wherein said ultraviolet radiation effects tanning of the skin; and
either manually rolling the apparatus or controlling forward and backward movement of the apparatus along a flat surface via a motorized drive system to focus the ultraviolet radiation on different parts of the body to achieve a complete tan or as desired.

9. A method of delivering light therapy comprising:
providing the apparatus of claim 1, 3 or 5;
lying on a flat surface underneath the apparatus;
emitting light from the plurality of bulbs for the purpose of therapeutic effect; and
wherein said apparatus is capable of being slid or rolled along a flat surface to affect light therapy at specific areas of the body.
